SEQUENCE LISTING (1 sequence)

SEQ ID NO: 1 - DNA, 18 bp, Artificial Sequence
  Synthetic
tctcccagcgtgcgccat

(19) United States
(12) Patent Application Publication
(10) Pub. No.: US 2020/0237678 A1
(43) Pub. Date: Jul. 30, 2020

(54) LIPID NANOPARTICLE MEMBRANE COMPOSITION

(71) Applicants: NANJING LUYE PHARMACEUTICAL CO., LTD., Jiangsu (CN); NANJING ASEC NANO BIOMEDICINE CO., LTD., Liangsu (CN)

(72) Inventors: Guang CHENG, Jiangsu (CN); Wenzhong CHEN, Jiangsu (CN); Lili QIN, Jiangsu (CN)

(21) Appl. No.: 16/342,804

(22) PCT Filed: Oct. 13, 2017
(86) PCT No.: PCT/CN2017/106032

§ 371 Date: Apr. 17, 2019

(30) Foreign Application Priority Data
Oct. 17, 2016 (CN) 201610902492.2

(51) Int. Cl. A61K 9/51; A61K 31/713

(52) U.S. Cl. CPC A61K 9/5123; A61K 31/713; A61K 9/5192; A61K 9/5146

(57) ABSTRACT

The present invention discloses a lipid nanoparticle membrane composition, and the membrane composition comprises a cationic lipid, a neutral phospholipid, cholesterol, Tween, and a polyethylene glycol derivative, with a molar ratio of (25-35):(40-50):(15-25):(1-5):(1-5) in the membrane composition. Also disclosed is a method for preparing a lipid nanoparticle from the lipid nanoparticle membrane composition. The present invention provides a lipid nanoparticle membrane composition that is capable of increasing the stability of the nanoparticle itself, thereby promoting the release of a medicament in tumor tissue and reducing the probability of being degraded.

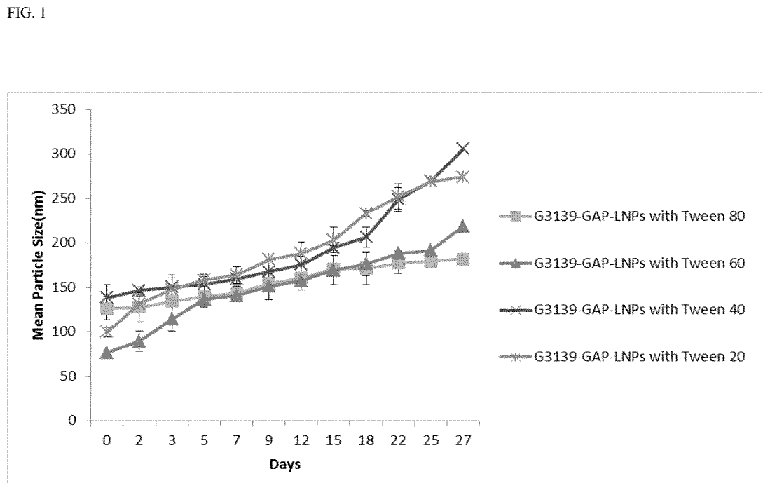

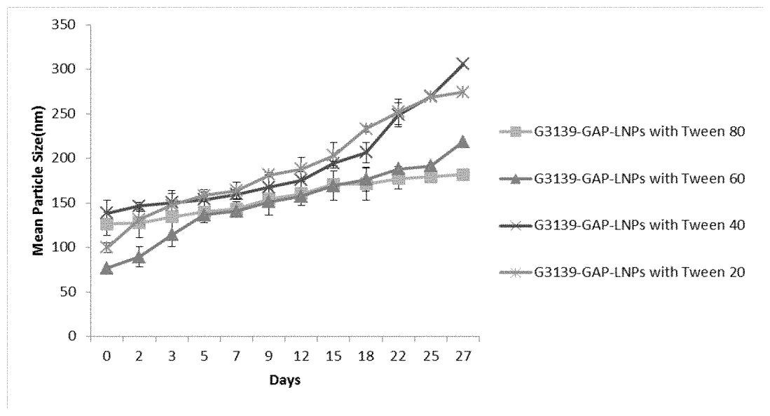

### TECHNICAL FIELD

The invention relates to the technical field of biology, in particular to a lipid nanoparticle membrane composition.

### BACKGROUND ART

There is no case where Tween is combined with a polyethylene glycol derivative such as TPGS in a lipid nanoparticle membrane of the prior art, for example, use of Tween: short-chain PEG alone, such that the short-chain PEG can increase the repellency between the nanoparticles to prevent the stability from being reduced due to their aggregation. On the other hand, due to the lack of long-chain PEG embedded on the surface of nanoparticles, this nano-preparation is easily phagocytosed by phagocytes and loses its function because Tween is easily lost in the systemic circulation.

Another example is the use of the polyethylene glycol derivative TPGS alone: although it has a long PEG chain, can prevent the recognition of the phagocyte system to a certain extent, does not lose its function for being phagocytosed by phagocytes, and increases the long circulation time. If there are a lot of TPGS, nanoparticles may be difficult to be effectively uptaken by target tumor cells, because of the steric-hindrance effect.

### SUMMARY OF THE INVENTION

### 1. Technical Problems to be Solved by the Present Invention

A purpose of the present invention is to overcome the above-mentioned technical deficiencies and provide a lipid nanoparticle membrane composition that is capable of increasing the stability of the nanoparticle itself, thereby promoting the release of a medicament in tumor tissue and reducing the probability of being degraded.

### 2. Embodiments

In order to achieve the above purpose, the embodiment provided by the present invention is as follows:

a lipid nanoparticle membrane composition, the membrane composition comprises a cationic lipid, a neutral phospholipid, cholesterol, Tween, and a polyethylene glycol derivative, with a molar ratio of (25-35):(40-50):(15-25):(1-5):(1-5) in the membrane composition.

In a further embodiment, the cationic lipid comprises: DOTAP, DOTMA, DDAB, and DODMA; the neutral phospholipid comprises: Egg PC, DOPC, DSPC, DPPC, and DMPC;

the Tween comprises: Tween 20, and Tween 40, Tween 60, Tween 80;
and the polyethylene glycol derivative comprises: mPEG-DPPE, mPEG-DMPE, mPEG-DSPE, mPEG-DMG TPGS, and mPEG-cholesterol.

In a further embodiment, the PEG in the polyethylene glycol derivative comprises monomethyl polyethylene glycol having a molecular weight of 550-5,000.

In a further embodiment, the PEG in the polyethylene glycol derivative comprises monomethyl polyethylene glycol having a molecular weight of 550, 750, 1,000, 2,000, 3,000, and 5,000.

In a further embodiment, the polyethylene glycol derivative is a substance formed by linking a hydrophilic polyethylene glycol chain or a methylated polyethylene glycol chain to a hydrophobic structure that can be embedded into a lipid bilayer; and the hydrophobic structure is cholesterol, vitamin E, dipalmitoylphosphatidylethanolamine, dimyristoylphosphatidylethanolamine, dimyristoylglycerol or a structural analogue.

In a further embodiment, the Tween is Tween 80.

A method for preparing a lipid nanoparticle from a lipid nanoparticle membrane composition, comprises the following steps:

(1) dissolving a cationic lipid, a neutral phospholipid, cholesterol, Tween, and a polyethylene glycol derivative in 80% ethanol in a certain molar ratio to obtain a mixed ethanol solution; dissolving a content in a PBS buffer to obtain a content mixed solution;

(2) mixing the resulting mixed ethanol solution and the content solution in equal volumes to obtain a 40% final ethanol concentration mixed solution;

(3) further diluting the 40% final ethanol concentration mixed solution obtained in step (2) with a PBS solution in an equal volume; repeatedly diluting the final ethanol concentration mixed solution with the PBS solution in an equal volume until a preparation mixed solution with a final ethanol concentration of less than 5% is obtained;

(4) adding a high-salinity solution to the preparation mixed solution obtained in step (3) to obtain a mixed solution containing high concentration salt;

(5) removing ethanol and free uncoated content in the mixed solution obtained in step (4) with an ultrafiltration or dialysis device; and

(6) filtering and sterilizing the product obtained in step (5) through a filter membrane or a filter element with a pore size of less than or equal to 0.22 μm to obtain the lipid nanoparticle.

In a further embodiment, the PBS buffer does not contain DNase and RNase, the specification of the PBS buffer in the step (1) is 1×pH=7, and the specification of the PBS buffer in the step (3) is 1×pH=7.4.

In a further embodiment, the high-salinity solution is a NaCl solution, and the concentration of NaCl in the mixed solution in step (4) is 0.1-1 M.

In a further embodiment, the concentration of NaCl in the mixed solution is 0.3-0.4 M, preferably 0.3 M.

In a further embodiment, the content is a nucleic acid.

In a further embodiment, the nucleic acid is an oligonucleotide.

In a further embodiment, the ultrafiltration or dialysis device has a molecular weight cutoff of 10,000 to 100,000 daltons.

In a further embodiment, the membrane composition is DOTAP, Egg PC, cholesterol, Tween 80, and TPGS in a molar ratio of 25:45:20:5:5.

In a further embodiment, the membrane composition is DOTMA, Egg PC, cholesterol, Tween 80, and TPGS in a molar ratio of 30:45:20:5:5.

In a further embodiment, the membrane composition is DOTAP, DSPC, cholesterol, Tween 80, and TPGS in a molar ratio of 30:50:20:5:5.

In a further embodiment, the membrane composition is DOTAP, Egg PC, cholesterol, Tween 80, and mPEG2000-DPPE in a molar ratio of 30:45:20:5:5.

DOTAP: 1,2-dioleoyl-3-trimethylaminopropane chloride salt

DOTMA: 1,2-dioleyloxy-3-trimethylaminopropane chloride salt

DDAB: dioctadecyldimethylammonium bromide

DODMA: 1,2-dioleyloxy-3-dimethylaminopropane

Egg PC: Egg Yolk Phosphatidylcholine

DOPC: Dioleoyl Phosphatidylcholine

DSPC: Distearoyl Phosphatidylcholine

DPPC: Dipalmitoyl Phosphatidylcholine

DMPC: Dimyristoyl Phosphatidylcholine

DPPE: Dipalmitoyl Phosphatidylethanolamine

DMPE: Dimyristoyl Phosphatidylethanolamine

TPGS: D-α-Tocopherol polyethylene glycol succinate

### 3. Beneficial Effects

Compared with the prior art, the present invention has the following significant advantages:

1. The nanoparticle membrane of the present invention uses a double PEGylation reagent (Tween and polyethylene glycol derivative) for the first time, which can increase the stability of the nanoparticle itself to a certain extent, promote the release of a medicament in tumor tissue and reduce the possibility of being degraded.

Tween is often used as a pegylation reagent in the preparation of a nano-preparation. The Tween series has short-chain PEG The Tween series such as Tween 20/40/60/80 can increase the repellency between nanoparticles in the nano-preparation, make them not easy to aggregate, can improve the stability of the nano-preparation to a certain extent, and has a small increase in particle size for long-term storage.

The polyethylene glycol derivative reagent comprises mPEG2000-DSPE and TPGS, which can be used as a component of another commonly used nano-preparation, has a long PEG chain which can promote the circulation of nanoparticles in the blood, preventing being uptake by reticuloendothelial cells or phagocytes and lose their function, can prevent the recognition of the immune system to a certain extent, and prolong the circulation time, and a longer PEG chain may influence the uptake of cells to a certain extent.

The present invention combines the two, the longer PEG chain and the shorter Tween, it was found that the composition of these two pegylation reagents can effectively improve the stability, the long circulation time, the release in target cells, and the ability to degrade genes in target cells of the nanoparticles coated with an antisense oligonucleotide in plasma.

2. The stability test of a composition of Tween series and TPGS at 4 degrees for 27 days according to the present invention, as shown in the sole FIGURE, shows that in the one-month cycle, the change of the particle size of the nano-preparation is small, indicating that Tween can maintain the overall stability of the preparation.

The specific experimental data are as follows: in the stability test of Tween 80 at 4 degrees for 27 days, the nano-preparation had a particle size changed from 126 nm to 181 nm, with the most significant stability;

in the stability test of Tween 20 at 4 degrees for 27 days, the nano-preparation had a particle size changed from 99.8 nm to 274.2 nm, with relatively good stability;

in the stability test of Tween 40 at 4 degrees for 27 days, the nano-preparation had a particle size changed from 138.5 nm to 305.9 nm, with relatively good stability;

and in the stability test of Tween 60 at 4 degrees for 27 days, the nano-preparation had a particle size changed from 76.7 nm to 218.6 nm, with relatively good stability.

3. The nano-liposome membrane composition in the present invention comprises a cationic lipid, a neutral phospholipid, cholesterol, Tween, and a polyethylene glycol derivative combined in a certain ratio, so that the prepared nano-liposome can effectively reduce costs, has good stability and high cell uptake, and is a very good nano-carrier.

4. The high-salinity solution added in the preparation process of the present invention can dissociate the free content adsorbed on the surface of the nanoparticles, such as oligonucleotide, and reduce the relatively large particle size generated due to the adsorption of the contents, and the dissociated contents are removed in the subsequent dialysis.

5. The preparation method of the lipid nanoparticles in the present invention adopts a stepwise dilution method with ethanol, however, the method of non-equal-volume on-line mixing used in the prior art is relatively complicated, and two pumps and different types of liquid storage tanks are required, which are not suitable for industrial production. Compared with the prior art, from the dynamics point of view, the present invention is more conducive to the dynamic balance of the two systems and promotes the stability of the subsequent suspension system.

### BRIEF DESCRIPTION OF THE DRAWINGS

The sole FIGURE is a graph showing the stability test of the composition of Tween series and TPGS at 4 degrees for 27 days according to the present invention.

### DETAILED DESCRIPTION OF THE INVENTION

The following describes the present invention and its embodiments schematically. The description is not restrictive. The drawings also show only one of the embodiments of the present invention. The actual structure is not limited thereto. Therefore, if those of ordinary skill in the art are inspired by it, without departing from the inventive concept of the present invention, to design a structure and an embodiment similar to the embodiment without creativity, it should belong to the protection scope of the present invention.

### Example 1

A lipid nanoparticle membrane composition, the membrane composition comprises DOTAP, Egg PC, cholesterol, Tween 80, and TPGS, with a molar ratio of 25:45:20:5:5 in the membrane.

A method for preparing a lipid nanoparticle from the above lipid nanoparticle membrane composition comprises the following steps:

(1) dissolving the above substances in 80% ethanol to obtain a mixed ethanol solution; dissolving an oligonucleotide G3139 in a PBS buffer (1×pH=7) to obtain a PBS solution of the oligonucleotide; G3139 is an antisense oligonucleotide consisting of 18 nucleotides, with a nucleotide sequence of 5′-TCT CCC AGC GTG CGC CAT-3′;

(2) mixing the obtained mixed ethanol solution and the PBS solution of the oligonucleotide G3139 in equal volumes to obtain a 40% final ethanol concentration mixed solution;

(3) further diluting the 40% final ethanol concentration mixed solution obtained in step (2) with a PBS solution in an equal volume; repeatedly diluting the final ethanol concentration mixed solution with a PBS solution (1×pH=7.4) in an equal volume until a preparation mixed solution with a final ethanol concentration of less than 5% is obtained;

(4) adding a high-salinity solution to the preparation mixed solution obtained in step (3) to obtain a mixed solution with a final concentration of 0.1 M;

(5) removing the ethanol and free antisense oligonucleotides in the mixed solution obtained in step (4) with an ultrafiltration device with a molecular weight cutoff of 10,000 daltons; and

(6) filtering and sterilizing the product obtained in step (5) through a filter membrane through a filter membrane with a pore size of 0.22 μm to obtain the nanoparticles.

### Example 2

A lipid nanoparticle membrane composition, the membrane composition comprises DOTMA, DOPC, cholesterol, Tween 40, and mPEG550-DPPE, with a molar ratio of 35:40:15:1:1 in the membrane.

A method for preparing a lipid nanoparticle from the above lipid nanoparticle membrane composition comprises the following steps:

(1) dissolving the above substances in 80% ethanol to obtain a mixed ethanol solution; dissolving an antisense oligonucleotide G3139 in a PBS buffer (1×pH=7) to obtain a PBS solution of the antisense oligonucleotide G3139;

(2) mixing the obtained mixed ethanol solution and the PBS solution of the antisense oligonucleotide G3139 in equal volumes to obtain a 40% final ethanol concentration mixed solution;

(3) further diluting the 40% final ethanol concentration mixed solution obtained in step (2) with a PBS solution in an equal volume; repeatedly diluting the final ethanol concentration mixed solution with a PBS solution (1×pH=7.4) in an equal volume until a preparation mixed solution with a final ethanol concentration of less than 5% is obtained;

(4) adding a high-concentration salt NaCl solution to the preparation mixed solution obtained in step (3) to obtain a mixed solution with a final concentration of 0.3 M;

(5) removing the ethanol and free antisense oligonucleotides in the mixed solution obtained in step (4) with an ultrafiltration device with a molecular weight cutoff of 50,000 daltons; and

(6) filtering and sterilizing the product obtained in step (5) through a filter membrane through a filter membrane with a pore size of 0.20 μm to obtain the nanoparticles.

### Example 3

A lipid nanoparticle membrane composition, the membrane composition comprises DDAB, DSPC, cholesterol, Tween 60, and mPEG2000-DMPE, with a molar ratio of 30:50:25:3:3 in the membrane.

The method for preparing a lipid nanoparticle from the above lipid nanoparticle membrane composition comprises the following steps:

(1) dissolving the above substances in 80% ethanol to obtain a mixed ethanol solution; dissolving an antisense oligonucleotide in a PBS buffer (1×pH=7) to obtain a PBS solution of the antisense oligonucleotide G3139;

(2) mixing the obtained mixed ethanol solution and the PBS solution of the antisense oligonucleotide G3139 in equal volumes to obtain a 40% final ethanol concentration mixed solution;

(3) further diluting the 40% final ethanol concentration mixed solution obtained in step (2) with a PBS solution in an equal volume; repeatedly diluting the final ethanol concentration mixed solution with a PBS solution (1×pH=7.4) in an equal volume until a preparation mixed solution with a final ethanol concentration of less than 5% is obtained;

(4) adding a high-salinity solution to the preparation mixed solution obtained in step (3) to obtain a mixed solution with a final concentration of 1 M;

(5) removing the ethanol and free antisense oligonucleotide G3139 in the mixed solution obtained in step (4) with an ultrafiltration device with a molecular weight cutoff of 100,000 daltons; and

(6) filtering and sterilizing the product obtained in step (5) through a filter membrane through a filter membrane with a pore size of 0.22 μm to obtain the nanoparticles.

### Example 4

(1) DOTAP/Egg PC/Cholesterol/Tween 80/TPGS=25:50:20:5:5

(2) DOTAP/Egg PC/Cholesterol/Tween 80/TPGS=30:50:20:5:5

(3) DOTAP/Egg PC/Cholesterol/Tween 80/TPGS=35:50:20:5:5

| | Particle size | Zeta potential | G3139 encapsulation rate |
|---|---|---|---|
| Preparation | (nm) | (mV) | (%) |
| 1 | 123.4 | 7.45 | 89.2% |
| 2 | 132.5 | 7.96 | 83.8% |
| 3 | 128.1 | 8.55 | 84.9% |

By adjusting the molar ratio of DOTAP-positive phospholipid and conducting single-factor experiments, it was found that the particle size and encapsulation rate of the first group were significantly better than those of the other two groups under the same conditions of other components, and the zeta potentials remained basically the same, and then the first group was selected as the optimal formula for the following screening.

(1) DOTAP/Egg PC/Cholesterol/Tween 80/TPGS=25:40:20:5:5

(2) DOTAP/Egg PC/Cholesterol/Tween 80/TPGS=25:45:20:5:5

(3) DOTAP/Egg PC/Cholesterol/Tween 80/TPGS=25:50:20:5:5

| | Particle size | Zeta potential | G3139 encapsulation rate |
|---|---|---|---|
| Preparation | (nm) | (mV) | (%) |
| 1 | 138.6 | 7.53 | 83.6% |
| 2 | 123.4 | 7.45 | 89.2% |
| 3 | 136.0 | 7.21 | 84.2% |

By adjusting the molar ratio of Egg PC-phospholipid and conducting single-factor experiments, it was found that the particle size and encapsulation rate of the second group were significantly better than those of the other two groups under the same conditions of other components, and the zeta potential remained basically the same, and then the second group was selected as the optimal formula for the following screening.

(1) DOTAP/Egg PC/Cholesterol/Tween 80/TPGS=25:45:15:5:5

(2) DOTAP/Egg PC/Cholesterol/Tween 80/TPGS=25:45:20:5:5

(3) DOTAP/Egg PC/Cholesterol/Tween 80/TPGS=25:45:25:5:5

| | Particle size | Zeta potential | G3139 encapsulation rate |
|---|---|---|---|
| Preparation | (nm) | (mV) | (%) |
| 1 | 120.4 | 7.78 | 80.4% |
| 2 | 123.4 | 7.45 | 89.2% |
| 3 | 143.4 | 7.03 | 86.2% |

By adjusting the molar ratio of cholesterol and conducting single-factor experiments, it was found that the particle size and encapsulation rate of the second group were significantly better than those of the other two groups under the same conditions of other components, and the zeta potential remained basically the same, and then the second group was selected as the optimal formula for the following screening.

(1) DOTAP/Egg PC/Cholesterol/Tween 80/TPGS=25:45:15:1:5

(2) DOTAP/Egg PC/Cholesterol/Tween 80/TPGS=25:45:20:3:5

(3) DOTAP/Egg PC/Cholesterol/Tween 80/TPGS=25:45:25:5:5

| | Particle size | Zeta potential | G3139 encapsulation rate |
|---|---|---|---|
| Preparation | (nm) | (mV) | (%) |
| 1 | 135.4 | 9.23 | 78.4% |
| 2 | 128.0 | 8.53 | 84.7% |
| 3 | 123.4 | 7.45 | 89.2% |

By adjusting the molar ratio of Tween 80 and conducting single-factor experiments, it was found that the particle size and encapsulation rate of the third group were significantly better than those of the other two groups under the same conditions of other components, and the zeta potential remained basically the same, and then the third group was selected as the optimal formula for the following screening.

(1) DOTAP/Egg PC/Cholesterol/Tween 80/TPGS=25:45:15:5:1

(2) DOTAP/Egg PC/Cholesterol/Tween 80/TPGS=25:45:20:5:3

(3) DOTAP/Egg PC/Cholesterol/Tween 80/TPGS=25:45:25:5:5

| | Particle size | Zeta potential | G3139 encapsulation rate |
|---|---|---|---|
| Preparation | (nm) | (mV) | (%) |
| 1 | 129.2 | 8.53 | 82.6% |
| 2 | 124.6 | 7.91 | 79.3% |
| 3 | 123.4 | 7.45 | 89.2% |

By adjusting the molar ratio of TPGS and conducting single-factor experiments, it was found that the particle size and encapsulation rate of the third group were significantly better than those of the other two groups under the same conditions of other components, and the zeta potential remained basically the same, and then the third group was selected as the optimal formula for the following screening.

(1) DOTMA/Egg PC/Cholesterol/Tween 80/TPGS=25:45:20:5:5

(2) DOTMA/Egg PC/Cholesterol/Tween 80/TPGS=30:45:20:5:5

(3) DOTMA/Egg PC/Cholesterol/Tween 80/TPGS=35:45:20:5:5

| | Particle size | Zeta potential | G3139 encapsulation rate |
|---|---|---|---|
| Preparation | (nm) | (mV) | (%) |
| 1 | 131.4 | 5.76 | 88.4% |
| 2 | 127.2 | 6.66 | 91.9% |
| 3 | 126.4 | 6.75 | 84.6% |

In addition, other phospholipids were also screened. By adjusting the molar ratio of DOTMA-positive phospholipid and conducting single-factor experiments, it was found that the particle size and encapsulation rate of the second group were significantly better than those of the other two groups under the same conditions of other components, and the zeta potential remained basically the same, and then the second group was selected as the optimal formula for the following screening.

(1) DOTAP/DSPC/Cholesterol/Tween 80/TPGS=30:40:20:5:5

(2) DOTAP/DSPC/Cholesterol/Tween 80/TPGS=30:45:20:5:5

(3) DOTAP/DSPC/Cholesterol/Tween 80/TPGS=30:50:20:5:5

| | Particle size | Zeta potential | G3139 encapsulation rate |
|---|---|---|---|
| Preparation | (nm) | (mV) | (%) |
| 1 | 124.4 | 7.12 | 82.8% |
| 2 | 126.0 | 8.04 | 87.1% |
| 3 | 131.8 | 7.71 | 89.9% |

By adjusting the molar ratio of DSPC-phospholipid and conducting single-factor experiments, it was found that the particle size and encapsulation rate of the third group were significantly better than those of the other two groups under the same conditions of other components, and the zeta potential remained basically the same, and then the third group was selected as the optimal formula for the following screening.

(1) DOTAP/Egg PC/Cholesterol/Tween 80/DPPE-mPEG2000=30:45:20:5:1

(2) DOTAP/Egg PC/Cholesterol/Tween 80/DPPE-mPEG2000=30:45:20:5:3

(3) DOTAP/Egg PC/Cholesterol/Tween 80/DPPE-mPEG2000=30:45:20:5:5

| | Particle size | Zeta potential | Encapsulation rate |
|---|---|---|---|
| Preparation | (nm) | (mV) | (%) |
| 1 | 142.6 | 7.68 | 85.2% |
| 2 | 134.5 | 6.63 | 87.3% |
| 3 | 135.8 | 7.90 | 91.4% |

By adjusting the molar ratio of DPPE-mPEG2000 and conducting single-factor experiments, it was found that the particle size and encapsulation rate of the third group were significantly better than those of the other two groups under the same conditions of other components, and the zeta potential remained basically the same.

After the screening of the compositions and ratios of the above formulas, it can be concluded that the formula with the composition and ratio of DOTAP/Egg PC/Cholesterol/Tween 80/TPGS=25:45:20:5:5 has a relatively small and stable particle size and encapsulation rate, as well as a relatively moderate positive zeta potential. In addition, DOTMA/Egg PC/Cholesterol/Tween 80/TPGS=30:45:20:5:5, DOTAP/D SPC/Cholesterol/Tween 80/TPGS=30:50:20:5:5, and DOTAP/Egg PC/Cholesterol/Tween 80/DPPE-mPEG2000=30:45:20:5:5 are also preferred ratios in the composition of other components in terms of particle size, zeta potential, encapsulation rate, and stability.

The stability test of a composition of Tween series and TPGS at 4 degrees for 27 days according to the present invention, as shown in the sole FIGURE, shows that in the one-month, the change of the particle size of the nano-preparation is small, indicating that Tween can maintain the overall stability of the preparation.

The specific experimental data are as follows: in the stability test of Tween 80 at 4 degrees for 27 days, the G3139-GAP nano-preparation had a particle size changed from 126 nm to 181 nm, with the most significant stability;

in the stability test of Tween 20 at 4 degrees for 27 days, the G3139-GAP nano-preparation had a particle size changed from 99.8 nm to 274.2 nm, with relatively good stability;

in the stability test of Tween 40 at 4 degrees for 27 days, the G3139-GAP nano-preparation had a particle size changed from 138.5 nm to 305.9 nm, with relatively good stability;

and in the stability test of Tween 60 at 4 degrees for 27 days, the G3139-GAP nano-preparation had a particle size changed from 76.7 nm to 218.6 nm, with relatively good stability.

1. A lipid nanoparticle membrane composition, wherein the membrane composition comprises a cationic lipid, a neutral phospholipid, cholesterol, Tween, and a polyethylene glycol derivative, with a molar ratio of (25-35):(40-50):(15-25):(1-5):(1-5) in the membrane composition.

2. The lipid nanoparticle membrane composition according to claim 1, wherein the cationic lipid comprises: DOTAP, DOTMA, DDAB, and DODMA;
the neutral phospholipid comprises: Egg PC, DOPC, DSPC, DPPC, and DMPC;
the Tween comprises: Tween 20, Tween 40, Tween 60, and Tween 80;
and the polyethylene glycol derivative comprises: mPEG-DPPE, mPEG-DMPE, mPEG-DSPE, mPEG-DMG, TPGS, and mPEG-cholesterol.

3. The lipid nanoparticle membrane composition according to claim 1, wherein the PEG in the polyethylene glycol derivative comprises monomethyl polyethylene glycol having a molecular weight of 550-5,000.

4. The lipid nanoparticle membrane composition according to claim 3, wherein the polyethylene glycol derivative is a substance formed by linking a hydrophilic polyethylene glycol chain or a methylated polyethylene glycol chain to a hydrophobic structure that can be embedded into a lipid bilayer; and the hydrophobic structure is cholesterol, vitamin E, dipalmitoylphosphatidylethanolamine, dimyristoylphosphatidylethanolamine, dimyristoylglycerol or a structural analogue.

5. The lipid nanoparticle membrane composition according to claim 2, wherein the Tween is Tween 80.

6. A method for preparing a lipid nanoparticle from a lipid nanoparticle membrane composition, wherein comprising the following steps:
(1) dissolving a cationic lipid, a neutral phospholipid, cholesterol, Tween, and a polyethylene glycol derivative in 80% ethanol in a certain molar ratio to obtain a mixed ethanol solution; dissolving a content in a PBS buffer to obtain a content mixed solution;
(2) mixing the resulting mixed ethanol solution and the content solution in equal volumes to obtain a 40% final ethanol concentration mixed solution;
(3) further diluting the 40% final ethanol concentration mixed solution obtained in step (2) with a PBS solution in an equal volume; repeatedly diluting the final ethanol concentration mixed solution with the PBS solution in an equal volume until a preparation mixed solution with an ethanol final concentration of less than 5% is obtained;
(4) adding a high-salinity solution to the preparation mixed solution obtained in step (3) to obtain a mixed solution containing high concentration salt;
(5) removing ethanol and free uncoated contents in the mixed solution obtained in step (4) with an ultrafiltration or dialysis device; and
(6) filtering and sterilizing a product obtained in step (5) through a filter membrane or a filter element with a pore size of less than or equal to 0.22 m to obtain the lipid nanoparticle.

7. The method for preparing a lipid nanoparticle from a lipid nanoparticle membrane composition according to claim 6, wherein the PBS buffer does not contain DNase and RNase, the PBS buffer in the step (1) has a specification of 1×pH=7, and the PBS buffer in the step (3) has a specification of 1×pH=7.4.

8. The method for preparing a lipid nanoparticle from a lipid nanoparticle membrane composition according to claim 6, wherein the high-salinity solution is a NaCl solution, and the concentration of NaCl in the mixed solution in step (4) is 0.1-1 M.

9. The method for preparing a lipid nanoparticle from a lipid nanoparticle membrane composition according to claim 8, wherein the concentration of NaCl in the mixed solution is 0.3-0.4 M.

10. The method for preparing a lipid nanoparticle from a lipid nanoparticle membrane composition according to claim 6, wherein the content is a nucleic acid.

11. The method for preparing a lipid nanoparticle from a lipid nanoparticle membrane composition according to claim 10, wherein the nucleic acid is an oligonucleotide.

12. The method for preparing a lipid nanoparticle from a lipid nanoparticle membrane composition according to claim 6, wherein the ultrafiltration or dialysis device has a molecular weight cutoff of 10,000 to 100,000 daltons.

13. The lipid nanoparticle membrane composition according to claim 2, wherein the membrane composition comprises DOTAP, Egg PC, cholesterol, Tween 80, and TPGS in a molar ratio of 25:45:20:5:5.

14. The lipid nanoparticle membrane composition according to claim 2, wherein the membrane composition comprises DOTMA, Egg PC, cholesterol, Tween 80, and TPGS in a molar ratio of 30:45:20:5:5.

15. The lipid nanoparticle membrane composition according to claim 2, wherein the membrane composition comprises DOTAP, DSPC, cholesterol, Tween 80, and TPGS in a molar ratio of 30:50:20:5:5.

16. The lipid nanoparticle membrane composition according to claim 2, wherein the membrane composition comprises DOTAP, Egg PC, cholesterol, Tween 80, and mPEG2000-DPPE in a molar ratio of 30:45:20:5:5.
